(19) United States
(12) Patent Application Publication
(10) Pub. No.: US 2009/0171328 A1
(43) Pub. Date: Jul. 2, 2009

(54) Surgical Console Display Operable to Provide a Visual Indication of a Status of a Surgical Laser

(76) Inventor: Christopher Horvath, Newport Beach, CA (US)

Correspondence Address: ALCON, IP LEGAL, TB4-8, 6201 SOUTH FREEWAY, FORT WORTH, TX, 76134, US

(21) Appl. No.: 12/329,683

(22) Filed: Dec. 8, 2008

Related U.S. Application Data
(60) Provisional application No. 61/017,142, filed on Dec. 27, 2007.

(51) Int. Cl. A61B 18/20; A61B 19/00

(52) U.S. Cl. 606/10; 606/130

(57) ABSTRACT

A surgical console display operable to provide a visual indication of status of a surgical laser or other surgical instrument or peripheral device coupled to the surgical console is provided in accordance with embodiments of the present invention. This facilitates the surgeon's management of a surgical procedure where a multitude of tasks and surgical control equipment are manipulated during an ocular surgery. The surgical console includes a processing module, an external interface, and a user interface having a display screen. The display screen specifically allows operators to view a status of peripheral devices and/or surgical instruments such as but not limited to a surgical laser.

### RELATED APPLICATIONS

This application claims priority to U.S. Patent Application No. 61/017,142 filed Dec. 27, 2007.

### TECHNICAL FIELD OF THE INVENTION

The present invention relates generally to surgical consoles systems and methods, and more particularly, a system and method to facilitate surgical procedures.

### BACKGROUND OF THE INVENTION

During modern surgery, particularly ophthalmic surgery, the surgeon uses a variety of pneumatic and electronically driven microsurgical hand pieces. The hand pieces are operated by a microprocessor-driven surgical console that receives inputs from the surgeon or an assistant by a variety of peripheral devices, such as foot pedal controllers, infrared remote control devices and menu-driven touch screens. One such microsurgical console is described in U.S. Pat. No. 5,455,766 (Scheller, et al.), the entire content of which is incorporated herein by reference. Surgical consoles allow surgeons to manually input surgical operating parameters and store these “customized” parameters in the console memory for future use. Prior art devices, however, all require that the operating parameters and methodologies be inputted manually using a keypad, touch screen or downloaded from another console that has had the parameters inputted manually.

The human eye can suffer a number of maladies causing mild deterioration to complete loss of vision. While contact lenses and eyeglasses can compensate for some ailments, ophthalmic surgery is required for others. Generally, ophthalmic surgery is classified into posterior segment procedures, such as vitreoretinal surgery, and anterior segment procedures, such as cataract surgery. More recently, combined anterior and posterior segment procedures have been developed.

The surgical instrumentation used for ophthalmic surgery can be specialized for anterior segment procedures or posterior segment procedures or support both. In any case, the surgical instrumentation often requires the use of associated consumables such as surgical cassettes, fluid bags, tubing, hand piece tips and so on. In some cases, a surgical console may house some or all of the associated surgical instrumentation and consumables and may provide a centralized system for monitoring and/or controlling the same.

The setup and operation of an ophthalmic surgical console can be quite complex as setting up a surgical instrumentation generally involves various electrical cables and pneumatic/fluidic tubing, etc. Various alerts may be associated with the operation of the ophthalmic surgical console. The operator (surgeon) requires a great deal of training to gain broad experience in both performing the procedure and using the surgical instrumentation. In particular it is difficult for surgeons to gain experience in the handling of complications that may arise during procedures. Training experience is generally limited by the number of cases available within a hospital on which the surgeons can gain surgical experience. This results in an increased risk of complications with surgical procedures and forces the treatment of rare cases to be handled by specialized practitioners.

### SUMMARY OF THE INVENTION

Embodiments of the present invention provide a surgical console display operable to provide a visual indication of a status of a surgical laser or other attached peripheral device. The surgical console includes a processing module, an external interface, and a user interface having a display screen. The display screen allows the operators to initialize the user interface and display screen, select a surgical procedure to be executed and perform the surgical procedure. The display screen specifically allows operators to view a status of peripheral devices and/or surgical instruments such as but not limited to a surgical laser. Surgical laser systems require the console to have a “standby” and “ready” mode for safety purposes when a surgical laser is coupled to the console. The surgical laser may only be fired after a user has initiated a transition from “standby” to “ready.” This transition often requires a time delay such as a minimum 2 second(s) time delay and the system may fall back to standby if it remains too long in a ready condition to prevent damage to the surgical laser. Previously a LED or status light indicated the status of the system.

Embodiments of the present invention may also utilize the entire control surface (i.e. a display screen such as an LCD screen) and/or an entire front panel wherein the background color may change depending on the mode of operation of the surgical laser. In particular this would be achieved by changing the background color of the display from a neutral color such as white for “standby” to a different color such as green for “ready.” Furthermore control knobs and other features associated with this console may change color in the same way to further facilitate the user's understanding of the status.

Other advantages of the present invention will become more apparent to one skilled in the art upon reading and understanding the detailed description of the preferred embodiments described herein with reference to the following drawings.

### BRIEF DESCRIPTION OF THE DRAWINGS

For a more complete understanding of the present invention and the advantages thereof, reference is now made to the following description taken in conjunction with the accompanying drawings in which like reference numerals indicate like features and wherein:

FIG. 1 is a perspective view of one surgical console that may be used with embodiments of the present invention;

FIG. 2 is a functional block diagram of one surgical console in accordance with embodiments of the present invention;

FIG. 3 is a perspective view of one surgical console in accordance with embodiments of the present invention;

FIG. 4 provides two views of a display screen or control panel in accordance with embodiments of the present invention; and

FIG. 5 provides a logic flow diagram associated with one embodiment present invention that allows operators to use the surgical console or attached surgical instruments during a surgical procedure or exercise.

### DETAILED DESCRIPTION OF THE INVENTION

Preferred embodiments of the present invention are illustrated in the FIGS., like numerals being used to refer to like and corresponding parts of the various drawings.

A surgical console display operable to provide a visual indication of status of a surgical laser or other surgical instrument or peripheral device coupled to the surgical console is provided in accordance with embodiments of the present invention. This surgical console may be used to facilitate ocular surgical procedures. This allows the surgical console to directly integrate supported surgical instruments and peripheral devices such as a surgical laser. This facilitates the operator's management of a surgical procedure where a multitude of tasks and surgical control equipment are manipulated during ocular surgery.

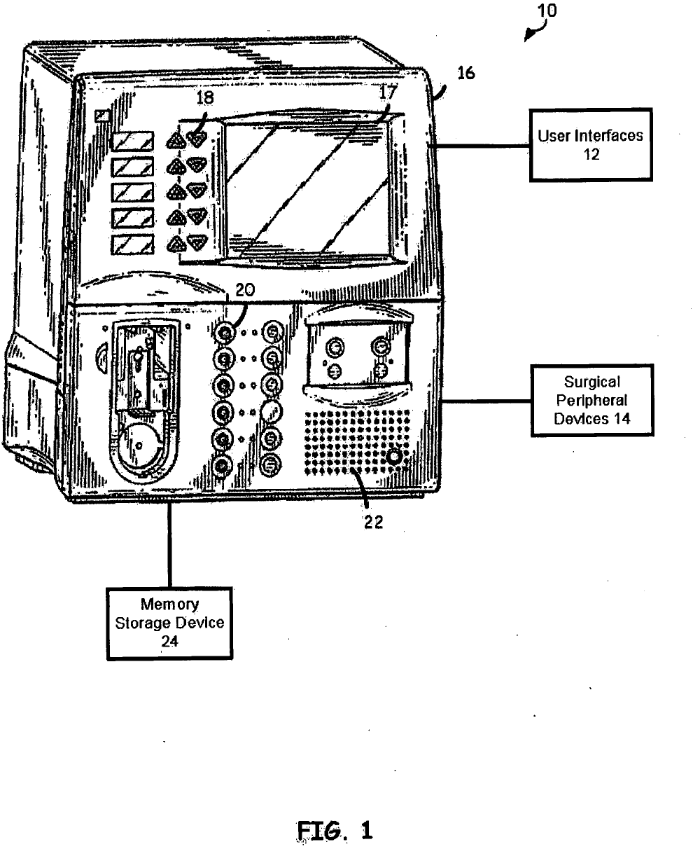

The device provided by embodiments of the present invention may be used with any suitable surgical console such as but not limited to the SERIES TWENTY THOUSAND® LEGACY®, the INFINITI® or the ACCURUS® surgical system consoles, as seen in FIG. 1, both commercially available from Alcon Laboratories, Inc., Fort Worth, Tex., that may be configured to support the use of training surgical procedures or individual exercises that help improve the operator's proficiency with the surgical console and peripheral devices coupled thereto.

FIG. 1 provides an illustration of a surgical console 10. Microsurgical console 10 may operably couple to a number of user interfaces 12 such as a foot pedal assembly or other push-button type assembly not shown and microsurgical peripheral devices 14. Console 10 allows an operator such as a surgeon to begin a surgical procedure by setting the initial operating parameters and modes into the console 10. This may be done by allowing the operator to interface with the surgical console 10 through user interfaces 12 or other interfaces provided on the front panel 16. These may include an electronic display screen 17, a plurality of push-button switches or touch-sensitive pads 18, the plurality of endless digital potentiometer knobs 20, or other like interfaces known to those skilled in the art. The push-button 18 and knobs 20 are actuable by an operator to access various different operating modes and functions used in various surgical parameters. Console 10 may also include the ability to accept storage media such as cassette tapes, memory cards, floppy disks, compact discs (CDs), digital video disks (DVDs), or other like devices known to those skilled in the art.

Electronic display screen 17 may be controlled by a processing module that allows the operator access to one or more different menus or messages which relate to the functions and operations of the various push buttons 18 and knobs 20. In one embodiment the display screen may be divided into display screen regions associated with individual buttons 18. This arrangement allows for the indicated function of each button 18 or knob 20 to be readily changed. Additionally the use of the electronic display screen 17 also permits the buttons 18 and knobs 20 to be labeled in virtually any language. The surgical console display 17 is operable to provide a visual indication of status of a surgical laser or other surgical instrument or peripheral device coupled to the surgical console 10 in accordance with embodiments of the present invention. This facilitates the surgeon's management of a surgical procedure where a multitude of tasks and surgical control equipment are manipulated during an ocular surgery. The surgical console 10 includes a processing module, an external interface, and a user interface having a display screen 17. The display screen 17 specifically allows operators to view a status of peripheral devices and/or surgical instruments such as but not limited to a surgical laser.

Microsurgical console 10 may be adapted for use with a number of different surgical instruments (i.e. microsurgical peripheral devices 14). For example, these may include a surgical laser, fiber optic illumination instrument, a surgical microscope, a vitrectomy unit, a fragmentation emulsification instrument, a cutting instrument, such as a guillotine cutter for vitrectomy procedures, micro-scissors inset for proportionate and multiple cutting. While the above-identified microsurgical instruments are provided for illustrative purposes it should be understood that the microsurgical console 10 can be used with other similar equipped instruments. The surgical console 10 can also be attached to similar training devices that perform these same functions. In such a case, the surgical console 10 can then coordinate a training surgical procedure for the integrated use of the peripheral devices or individual exercises (or games) that focus on specific piece(s) of equipment.

In general, any microsurgical instruments that are actuated or controlled by pneumatic or electronic signals may be operably coupled to and controlled by console 10. This control or actuation may be governed by pneumatic, electronic, optical, or other like signals known to those skilled in the art wherein the signals are generated by console 10. Each of these illustrated microsurgical devices that couple to console 10 may have different modes of operation that may require different settings or parameters that are provided by the microsurgical console 10. By saving these operating parameters and surgical modes which are associated with specific steps of a surgical procedure in memory the setup of the microsurgical peripheral devices is facilitated by eliminating the often tedious or cumbersome process of initializing these devices manually via the surgical console for each step of the surgical procedure.

Embodiments of the present invention facilitate the surgical procedure and help to reduce the risks to patients. Recorded surgical procedures facilitate pertinent changes to the operating modes and peripheral device operating parameters from console memory to initialize or setup the microsurgical devices for individual steps within an overall surgical procedure. At the completion of a surgical procedure the completed surgical procedure may be saved as a recorded procedure in memory coupled to console 10. It should be noted that within surgical console 10 is a processing module coupled to memory where the processing module is operable to execute the steps that will be discussed in the logic flow diagrams. These steps are accessed by the processing module in which the instructions or steps are stored as well as the recorded surgical procedures.

Surgical console 10 is operable to generate realistic surgical situations or appropriate exercises that enable trainees to become familiar with the operation of the surgical console and surgical instruments used during complex surgical procedures. These training surgical procedures or exercises may be varied in order to account for potential complications associated with the various procedures. This allows operators (surgeons) to become familiar and gain experience with the surgical instruments and use the surgical training procedures without the risk to the patient. The surgical console in addition to providing simulations may record the operating parameters during the training surgical procedure such that the surgical procedure may be critiqued and the surgeon's abilities can be assessed objectively.

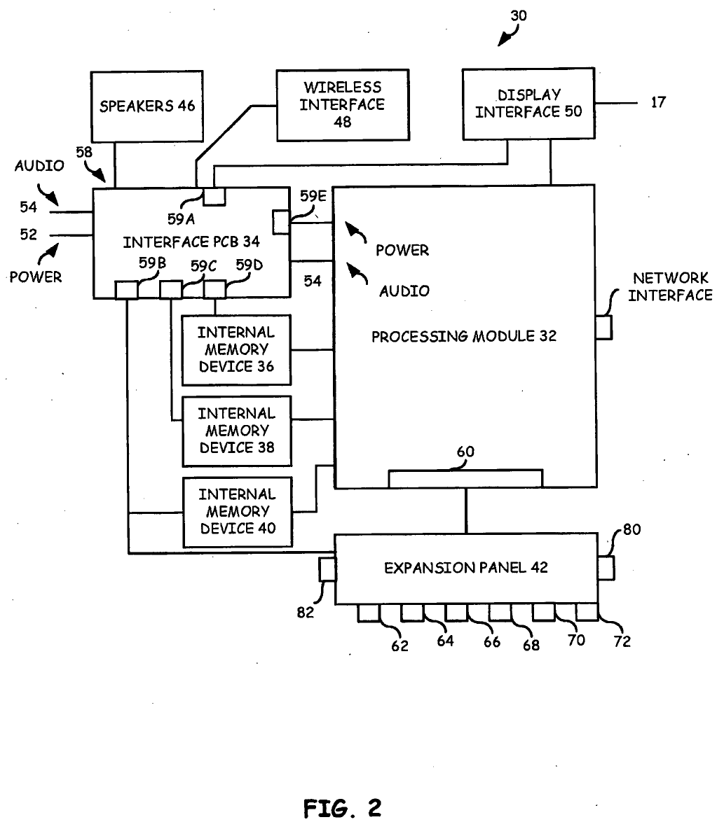

FIG. 2 depicts a block diagram of various functional modules that may be located within surgical console 10. This surgical console 10 may functionally include a processing system 32, a power signal 52 provided to input-output (I/O) interface printed circuit board (PCB) 34, mass storage devices 36, 38, and 40, speaker(s) 46, display port or connectors 50, expansion panel 42, and an external connection to audio inputs. Interface PCB 34 may include an audio output 58, a power output 59, and audio input 54. Interface PCB 34 couples to an external or internal power supply 52. Then interface PCB 34 may distribute power to various other elements contained within various other functional elements of surgical console 10. For example, power may be distributed through connections 59A, 59B, 59C, 59D and 59E to processing system 32, mass storage devices 36-40, expansion panels 42, and other functional units within the surgical console 10 as required. Additionally, interface PCB 34 may receive audio signals through audio ports 54 that may be for external connections with which to receive audio signals, or connections to processing system 32 that provides audio signals that interface PCB 34 may route to audio output port 58 and speakers 46. Mass storage devices 36-40 may further include hard drives, DVD drives, CD drives, and other like drives. Power supplied by the interface PCB 34 to these mass storage devices, wherein the multimedia content contained therein or other information contained therein may be accessed through various interfaces to processing module 32 and then routed to an appropriate playback portion of the surgical console through interface PCB 34. For example the audio signal may be routed to a speaker 46 in the case of a digital audio file such as an MP3 file, wave file or other like file or to display module 17 in the case of video or image content. Thus, interface PCB 34 with internal mass storage devices containing multi-media files to be played back during a surgical procedure or an external multi-media playback device such as, but not limited to, an MP3 player, may be coupled to the interface and provide audio and/or video signals to the interface which may then be processed using processing system 32 and presented using the appropriate playback means such as speakers 46 or display module 17. Additionally, control devices such as a keyword, mouse may be coupled to interface 34 in order to control the playback of the multi-media files. Otherwise, native controls such as buttons 18 and 20 which may have functions defined as presented in display 17, may be used to control the playback of the multi-media content.

The processing module 32 may be a single processing device or a plurality of processing devices. Such a processing device may be a microprocessor, micro-controller, digital signal processor, microcomputer, central processing unit, field programmable gate array, programmable logic device, state machine, logic circuitry, analog circuitry, digital circuitry, and/or any device that manipulates signals (analog and/or digital) based on operational instructions. The memory 60 may be a single memory device or a plurality of memory devices. Such a memory device may be a read-only memory, random access memory, volatile memory, non-volatile memory, static memory, dynamic memory, flash memory, cache memory, and/or any device that stores digital information. Note that when the processing module 32 implements one or more of its functions via a state machine, analog circuitry, digital circuitry, and/or logic circuitry, the memory 60 storing the corresponding operational instructions may be embedded within, or external to, the circuitry comprising the state machine, analog circuitry, digital circuitry, and/or logic circuitry. The memory 60 stores, and the processing module 32 executes, operational instructions corresponding to at least some of the steps and/or functions illustrated in the FIGS.

Descriptions of known programming techniques, computer software, hardware, operating platforms and protocols may be omitted so as not to unnecessarily obscure the invention in detail. It should be understood, however, that the detailed description and the specific examples, while indicating the preferred embodiments of the invention, are given by way of illustration only and not by way of limitation. Various substitutions, modifications, additions and/or rearrangements within the spirit and/or scope of the underlying inventive concept will become apparent to those skilled in the art from this disclosure.

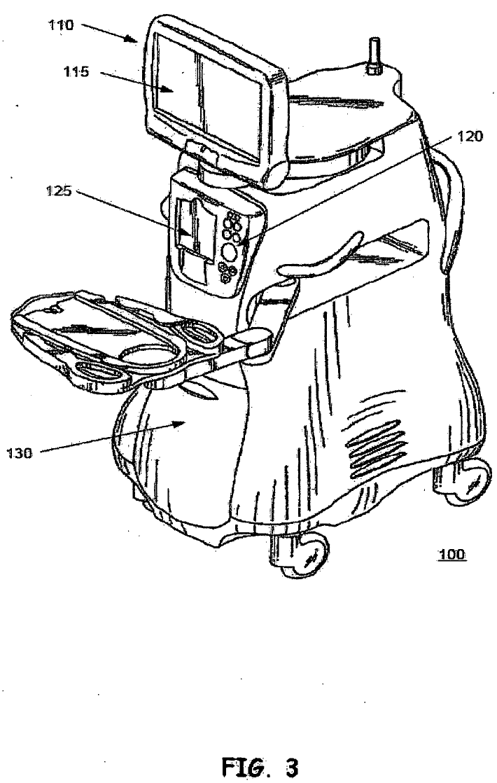

FIG. 3 is a diagrammatic representation of one embodiment of an ophthalmic surgical console 100. Surgical console 100 can include a swivel monitor 110 that has touch screen 115. Swivel monitor 110 can be positioned in a variety of orientations for whomever needs to see touch screen 115. Swivel monitor 10 can swing from side to side, as well as rotate and tilt. Touch screen 115 provides a graphical user interface (“GUI”) that allows a user to interact with console 100.

Surgical console 100 also includes a connection panel 120 used to connect various tools and consumables to surgical console 100. Connection panel 120 can include, for example, a coagulation connector, balanced salt solution receiver, connectors for various hand pieces and a fluid management system (“FMS”) or cassette receiver 125. Surgical console 100 can also include a variety of user friendly features, such as a foot pedal control (e.g., stored behind panel 130) and other features.

In operation, a cassette (not shown) can be placed in cassette receiver 125. Clamps in surgical console 100 clamp the cassette in place to minimize movement of the cassette during use. The clamps can clamp the top and bottom of the cassette, the sides of the cassette or otherwise clamp the cassette.

Surgical console 100 is provided by way of example and embodiments of the present invention can be implemented with a variety of surgical systems. Example surgical systems in which cassettes according to various embodiments of the present invention can be used include, for example, the Series 2000® Legacy® cataract surgical system, the Accurus® 400VS surgical system, and the Infiniti™ Vision System surgical system, all available from Alcon Laboratories Inc. of Fort Worth, Tex. Additionally, embodiments of the present invention can be used with a variety of surgical cassettes, examples of which are described in U.S. Pub. Nos. 2005/0186098 (application Ser. No. 11/114,289 to Davis et al.), 2004/0253129 (application Ser. No. 10/891,642 to Sorensen et al.), 2005/0065462 (application Ser. No. 10/979,433 to Nazarifar et al.), 2003/0225363 (application Ser. No. 10/156,175 to Gordon et al.), 2001/0016711 (application Ser. No. 09/846,724 to Sorensen et al.) and U.S. Pat. No. 6,293,926 to Sorensen et al., U.S. Pat. No. 4,493,695 to Cook, U.S. Pat. No. 4,627,833 to Cook, 4,395,258 to Wang et al., U.S. Pat. No. 4,713,051 to Steppe, et al., U.S. Pat. No. 4,798,850 to Brown, U.S. Pat. No. 4,758,238 to Sundblom et al., U.S. Pat. No. 4,790,816 to Sundblom et al., U.S. Pat. No. 6,267,956 to Gomes, et. al., U.S. Pat. No. 6,364,342 to Kim, 6,036,458 to Cole et al., and U.S. Pat. No. 6,059,544 to Jung et al., each of which is hereby fully incorporated by reference herein. Embodiments of the present invention can be implemented for other suitable surgical systems and cassettes as would be understood by one of ordinary skill in the art.

The status of various peripheral devices and or surgical instruments during the surgical procedure may be provided on the console screen. The display may be directly integrated with the actual surgical instruments that the operator (doctor) uses to perform an actual surgery.

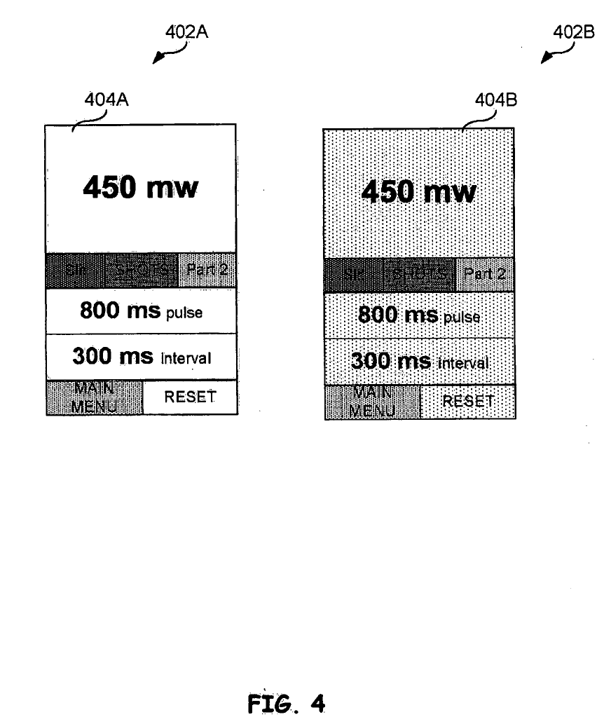

FIG. 4 provides two views of a display screen 402A and 402B in accordance with embodiments of the present invention. Display screen 402A and 402B may be created with the electronic display 17 of FIG. 1, display interface 50 of FIG. 2, touch screen 115 of FIG. 3. This display screen may be a liquid crystal display (LCD) or other like device known to those having skill in the art. As shown in 402A, information associated with the standby condition (mode or status) of a surgical laser is provided. The neutral or white background 404A is provided in example 402A. Information associated with this display may include a power level associated with the surgical laser, the number of shots (i.e. laser pulses), the time duration of the laser pulse, and a time period between laser pulses. In 402B, a darkened background 404B indicates a change in the status of the surgical laser from that shown in example 402A. For example, the background 404A background color may change from a light or neutral color in example 402A to a dark color in 402B. For example, this change may be from a white to a green or red or other color wherein the color may be chosen by the users based on their preferences. Additionally, lights or illuminated knobs and buttons on the user interface may change colors such that not only the background of the liquid crystal display would change, but all background colors associated with the display may change to indicate a change in status or mode of operation of a peripheral or surgical instrument, such as a surgical laser. This may indicate going from a standby to a ready condition.

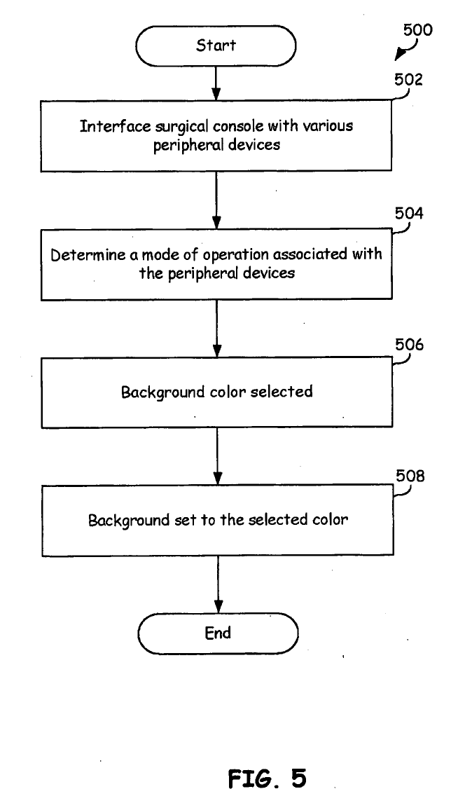

FIG. 5 provides a logic flow diagram associated with the embodiments of the present invention that allows operators to use the surgical console and attached surgical instruments during a surgical procedure. Operations 500 begin at step 502 where a surgical console may be interfaced with various peripheral devices and surgical instruments such as, but not limited to, a surgical laser. In step 504, the surgical console determined a mode of operation associated with the peripheral device, i.e., surgical laser. In step 506, a color for the background of the display screen and/or user interface is selected based on the mode of operation identified in step 504. In step 508, the background is set to a selected color in order to provide operators a clear indication of the status or mode of operation of the surgical instruments coupled to the device. There may be a transition wherein when a peripheral device or surgical instrument is transitioning from one mode or status to another that the background of the display screen or a user interface may alternate in color between that of the two end point states or modes of operation. Alternatively, a third color, such as yellow, may be selected to indicate that a transitory condition exists and that the peripheral device or surgical instrument is not available for use.

Embodiments of the present invention allow operators to handle a multitude of tasks and control a multitude of surgical equipment during ocular surgery. The background display ability to change colors as provided by embodiments of the present invention allows operators to remain focused on the surgical procedure in order to avoid accidental injury to a patient during ocular surgery. Furthermore, this allows operators to more easily be informed about the status of peripheral devices and systems coupled to the surgical console and may eliminate the need for an operator to have an individual verify the status on the surgical console by allowing the primary operator to determine that information with a mere glance at the front panel of the surgical console, even when the surgical console may be located at a distance from the primary operator.

In summary, a surgical console display operable to provide a visual indication of status of a surgical laser or other surgical instrument or peripheral device coupled to the surgical console is provided in accordance with embodiments of the present invention. A surgical console may be used to facilitate ocular surgical procedures. This allows the surgical console to directly integrate supported surgical instruments and peripheral devices such as a surgical laser. This facilitates the surgeon's management of a surgical procedure where a multitude of tasks and surgical control equipment are manipulated during ocular surgery.

The surgical console provided includes a processing module, an external interface, and a user interface having a display screen. The display screen allows the operators to initialize the user interface and display screen, select a surgical procedure to be executed and perform the surgical procedure. The display screen specifically allows operators to view a status of peripheral devices and/or surgical instruments such as but not limited to a surgical laser. Surgical laser systems require the console to have a “standby” and “ready” mode for safety purposes when a surgical laser is coupled to the console. The surgical laser may only be fired after a user has initiated a transition from “standby” to “ready.” This transition often requires a time delay such as a minimum 2 second(s) time delay and the system may fall back to standby if it remains too long in a ready condition to prevent damage to the surgical laser. Previously a LED or status light indicated the status of the system.

Embodiments of the present invention may utilize the entire control surface (i.e. a display screen such as an LCD screen) and/or an entire front panel wherein the background color may change depending on the mode of operation of the surgical laser. In particular this would be achieved by changing the background color of the display from a neutral color such as white for “standby” to a different color such as green for “ready.” Furthermore control knobs and other features associated with this console may change color in the same way to further facilitate the user's understanding of the status.

As one of average skill in the art will appreciate, the term “substantially” or “approximately”, as may be used herein, provides an industry-accepted tolerance to its corresponding term. Such an industry-accepted tolerance ranges from less than one percent to twenty percent and corresponds to, but is not limited to, component values, integrated circuit process variations, temperature variations, rise and fall times, and/or thermal noise. As one of average skill in the art will further appreciate, the term “operably coupled”, as may be used herein, includes direct coupling and indirect coupling via another component, element, circuit, or module where, for indirect coupling, the intervening component, element, circuit, or module does not modify the information of a signal but may adjust its current level, voltage level, and/or power level. As one of average skill in the art will also appreciate, inferred coupling (i.e., where one element is coupled to another element by inference) includes direct and indirect coupling between two elements in the same manner as “operably coupled”. As one of average skill in the art will further appreciate, the term “compares favorably”, as may be used herein, indicates that a comparison between two or more elements, items, signals, etc., provides a desired relationship. For example, when the desired relationship is that signal 1 has a greater magnitude than signal 2, a favorable comparison may be achieved when the magnitude of signal 1 is greater than that of signal 2 or when the magnitude of signal 2 is less than that of signal 1.

Although the present invention is described in detail, it should be understood that various changes, substitutions and alterations can be made hereto without departing from the spirit and scope of the invention as described by the appended claims.

What is claimed is:

1. A surgical console, comprising:
a processing module operable to direct operations of and receive inputs from peripheral devices operably coupled to the surgical console, wherein these peripheral devices comprise surgical instruments;
at least one memory device operably coupled to the processing module; and
a display screen coupled to the processing module, wherein the display screen allows operators to view a status of the peripheral devices.

2. The surgical console of claim 1, wherein a color change of the display screen allows operators to view a status of the peripheral devices.

3. The surgical console of claim 1, wherein the peripheral devices comprise a surgical laser.

4. The surgical console of claim 3, wherein the color change indicates a mode of operation of the surgical laser.

5. The surgical console of claim 1, further comprising an external interface operably coupled to the processing module, wherein the external interface is operable to interface the surgical console to the surgical instruments.

6. The surgical console of claim 1, wherein the peripheral devices comprise a surgical microscope.

7. The surgical console of claim 1, wherein the display screen comprises a LCD display.

8. A method for performing a surgical procedure using a surgical console, comprising:
interfacing the surgical console with various peripheral devices, wherein the peripheral devices comprise surgical instruments;
selecting the surgical procedure to be performed;
initializing the surgical console and the peripheral devices for the surgical procedure; and
performing the surgical procedure, wherein:
a display screen of the surgical console changes colors and wherein the colors displayed are each associated with a status of the peripheral devices.

9. The method of claim 8, wherein the peripheral devices comprise a surgical laser.

10. The method of claim 8, wherein a color change indicates a change in status of the surgical instruments.

11. The method of claim 8, wherein the surgical console further comprises an external interface operably coupled to the processing module, wherein the external interface is operable to interface the surgical console to the surgical instruments.

12. The method of claim 8, wherein the peripheral devices comprise a surgical microscope.

13. The method of claim 8, wherein the display screen comprises a LCD display.

14. The method of claim 8, wherein the surgical procedure is an ophthalmic surgical procedure.

15. A surgical console, comprising:
a processing module operable to:
direct operations of peripheral devices including surgical instruments operably coupled to the surgical console; and
monitor operating parameters and surgical modes associated with a surgical procedure;
an external interface operably coupled to the processing module, wherein the external interface is operable to interface the surgical console to the surgical instruments;
a user interface, comprising a display screen, wherein the user interface allows operators to:
initialize the surgical console for the surgical procedure;
select the surgical procedure to be executed; and
perform the surgical procedure, wherein the display screen allows operators to view a status of the peripheral devices wherein the surgical instruments comprise a surgical laser

16. The surgical console of claim 15, wherein a color change of the display screen allows operators to view the status of the peripheral devices.

17. The surgical console of claim 15, wherein the color change indicates a mode of operation of the surgical laser.

18. The surgical console of claim 15, wherein the display screen comprises a LCD display.

19. The surgical console of claim 15, wherein the surgical procedure is an ophthalmic surgical procedure.
